(19) United States
(12) Patent Application Publication
(10) Pub. No.: US 2023/0414679 A1
(43) Pub. Date: Dec. 28, 2023

(54) CONTROL OF HISTAMINE TO PROMOTE HEALTH AND CONTROL ENTEROCOLITIS USING PROBIOTIC COMPOSITIONS

(71) Applicant: Iowa State University Research Foundation, Inc., Ames, IA (US)

(72) Inventor: Mark Lyte, Ames, IA (US)

(21) Appl. No.: 18/339,806

(22) Filed: Jun. 22, 2023

Related U.S. Application Data
(60) Provisional application No. 63/366,809, filed on Jun. 22, 2022.

(51) Int. Cl. A61K 35/742; A61K 38/44; A61P 37/06

(52) U.S. Cl. CPC A61K 35/742; A61K 38/44; A61P 37/06; C12R 2001/125

(57) ABSTRACT

The present disclosure relates to use of probiotic compositions and/or histamine degrading enzymes for the control of histamine in a farm production animal, companion animal, aquaculture, or a human, to reduce or eliminate the incidence of necrotizing enterocolitis and/or related inflammatory conditions in the gastrointestinal tract or skin, and behavioral conditions. The disclosure also relates to the probiotic compositions and/or histamine degrading enzymes.

### CROSS REFERENCE TO RELATED APPLICATIONS

This application claims priority under 35 U.S.C. § 119 to provisional patent application U.S. Ser. No. 63/366,809, filed Jun. 22, 2022. The provisional patent application is herein incorporated by reference in its entirety, including without limitation, the specification, claims, and abstract, as well as any figures, tables, appendices, or drawings thereof.

### TECHNICAL FIELD

The present disclosure relates generally to the use of probiotic compositions containing histamine degrading bacterial species to promote health and treat and/or prevent disease.

### BACKGROUND

There is ongoing understanding on the role of commensal microbiota and their influence on behavior, health, and disease susceptibility. Commensal or commensalism refers to a relationship between organisms that feed from each other in a way that neither benefits nor harms the relationship of the microbiota. Infections in farm production animals, companion animals, aquaculture and humans can cause significant damage or behavioral changes to the animals/humans and cause significant economic harm in the instance of animal producers. Various infections and inflammatory conditions within the gut of these species, namely farm production animals, is a multi-billion-dollar problem within the animal food production industry.

According to a 2012 Animal and Plant Health Inspection Service USDA report, 42.3 percent of turkey farms have problems with clostridial dermatitis (CD). CD can cause swelling, fluid accumulation, and vesicles on the subcutaneous breast region. Turkeys with this condition usually die; the 2012 report highlights the mortality rate of 4-17% due to CD outbreaks. CD is a disease of turkeys having significant economic impact on turkey producers across various geographic regions. Its prevalence and severity continues to increase and it has been identified as the most significant disease facing the turkey industry. The significant economic losses that turkey farmers have to bear result from bird mortality at marketable age, increased condemnation rates, and expensive medication costs for treatment. Economic losses ascribed to CD were projected to be $1.31 per affected bird. CD is a major health problem in commercial poultry raised on deep litter systems as are customary in US farms, however no known mechanisms other than the involvement of Clostridial spp. is known and more importantly no known effective treatments are known.

Similar to the deep litter systems used in turkey farms, the use of hardwood shavings as litter in chicken coops has been found to correlate with higher incidence of cellulitis. Moreover, survival of poultry pathogens in built-up litter is known to contribute to persistence and carry-over of related diseases to subsequent flocks in the barns. There has been little study into the impact of environmental risk factors on the incidence of CD in turkeys or the incident in cellulitis in chickens. However, the problems in the industry, causing reductions in avian farm production animals have gone unsolved.

However, bacterial infections are not the only way inflammatory or behavioral conditions can develop. As many of the microbiota, especially Clostridial species, are normal members of the gut microbiota (as described herein as commensal bacteria) in farm production animals as well as humans. As such, inflammation or behavioral conditions may result from dysregulation of the normal microbial diversity (such as by stress or other factor) thereby allowing those members of the microbiota to either become greater in number (abundance) or produce more histamine. Accordingly, there is a need for preventing/treating not only infections but also controlling effects of members of the microbiota that can cause increases in histamine production.

### SUMMARY

Methods of controlling histamine production in a subject are provided. In certain embodiments, the method comprises administering a probiotic composition comprising a Brevibacillus spp. and/or Bacillus spp. and optionally a histamine degrading enzyme to the subject and controlling histamine production within the subject.

Methods of preventing and/or treating an inflammatory disease and/or immunological condition mediated by histamine in a subject are also provided. In certain embodiments, the method comprises administering a probiotic composition comprising a Brevibacillus spp. and/or Bacillus spp. and optionally a histamine degrading enzyme to the subject and controlling histamine production within the subject.

Probiotic compositions comprising a therapeutically effective amount of a Brevibacillus spp. and/or Bacillus spp. and a therapeutically effective excipient, additional functional ingredient, and/or a food/feed component are also provided.

The foregoing summary is illustrative only and is not intended to be in any way limiting. In addition to the illustrative aspects, embodiments, and features described above, further aspects, embodiments, and features will become apparent by reference to the drawings and the following detailed description. Accordingly, the drawings and detailed description are to be regarded as illustrative in nature and not restrictive.

### BRIEF DESCRIPTION OF THE DRAWINGS

FIG. 1 shows the chemical reaction of a histamine-pyruvate aminotransferase transferring an amine from histamine to pyruvate and in the process generating alanine.

FIG. 2 shows a graphical example of the complete degradation of histamine in B. borstelenis isolates from 11 chickens sampled that each received either low (5 mg) or high (15 mg) histamine diets (mg histamine/100 grams feed) compared to a Control Medium (histamine minimal medium supplemented with 10 mM histamine in the absence of added bacteria).

FIG. 3 shows that a 2-day culture of Bacillus flexus in ⅓rd concentration of Luria-Bertani (LB) medium supplemented with 10 mM histamine was able to completely degrade the histamine. “Media Control” was ⅓rd LB supplemented with 10 mM histamine in the absence of added bacteria. The control did not evidence any degradation of histamine over the incubation period. Cultures of B. flexus were able to achieve 100% degradation of histamine as shown by the symbol “#”. Histamine was measured by ultra-high-performance liquid chromatography (UHPLC) methodology.

FIG. 4 shows that a 2-day culture of Bacillus subtilis in Histamine Minimal Medium (HMM) containing 10 mM histamine was able to degrade nearly 50% of the histamine present in the medium. “Media Control” was HMM supplemented with 10 mM histamine in the absence of added bacteria. The control did not evidence any degradation of histamine over the incubation period. Histamine was measured by UHPLC methodology.

Reference to various embodiments does not limit the scope of the disclosure. Figures represented herein are not limitations to the various embodiments according to the disclosure and are presented for exemplary illustration of the disclosure.

### DETAILED DESCRIPTION

The present disclosure relates to probiotic compositions and methods of treating a subject with a probiotic composition and/or histamine degrading enzymes. The embodiments are not limited to particular methods and compositions depicted herein, which can vary and may be understood by skilled artisans. It is further to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

In order to provide a clear and consistent understanding of the specification and the claims, including the scope given to such terms, the following definitions are provided. Units, prefixes, and symbols may be denoted in their SI accepted form.

The term “about,” as used herein, refers to variations in size, distance or any other types of measurements that can be resulted from inherent heterogeneous nature of the measured objects and imprecise nature of the measurements itself. The term “about” also encompasses variation in the numerical quantity that can occur, for example, through typical measuring and liquid handling procedures used for making media and reagents; through inadvertent error in these procedures; through differences in the manufacture, source, or purity of the ingredients used to make the compositions or carry out the methods, and moreover may modify the typical measurements referenced herein, and the like. Whether or not modified by the term “about”, the claims include equivalents to the quantities.

As used herein, the term “administering” refers to providing the probiotic or synbiotic composition to the subject. In one embodiment, the subject is provided the probiotic or synbiotic composition internally, and/or the administration of histamine-degrading enzyme either coated to protect activity, or alone, or as part of a mash, by a method or route which results in at least partial localization of the compound or composition to the gut or other hollow organ (e.g. oral cavity) such that a desired effect is produced. A composition described herein can be administered to the subject by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

In another embodiment, the probiotic or synbiotic compositions may be provided “topically” to the skin. As used herein, the term “topically” includes providing the compositions externally or by a shallow injection under the skin.

As used herein, an “effective amount” or “therapeutically effective amount” refers to the amount of the probiotic composition and/or histamine degrading enzymes that is sufficient to prevent, treat, reduce and/or ameliorate the symptoms and/or underlying causes of a disorder or disease. In an exemplary aspect, an “effective amount” or “therapeutically effective amount” refers to the amount of probiotic that is sufficient to prevent, inhibit, and/or treat clostridial dermatitis, clostridial enteric disease, and/or gut inflammation in the gut of the subject, including farm production animals, companion animals, aquaculture, and humans.

Also, as used herein, the term “gut” refers to the gastrointestinal tract as well as the liver, spleen, pancreas, omentum, and other organs served by the blood supply to and from the gut.

The term “microbiome”, as used herein, refers to a population of microorganisms from a particular environment, including the environment of the body or a part of the body. The term is interchangeably used to address the population of microorganisms itself (sometimes referred to as the microbiota), as well as the collective genomes of the microorganisms that reside in the particular environment. The term “environment,” as used herein, refers to all surrounding circumstances, conditions, or influences to which a population of microorganisms is exposed. The term is intended to include environments in a subject, such as a bird. Specifically, the term “intestinal microbiota”, as used herein, refers to the population of microorganisms inhabiting the gastrointestinal tract. The term was previously referred to as the intestinal flora. The term “skin microbiota”, as used herein, refers to the population of microorganisms inhabiting the skin.

“Microorganism” refers to an organism or microbe of microscopic, submicroscopic, or ultramicroscopic size that typically consists of a single cell. Examples of microorganisms include bacteria, viruses, parasites, fungi, certain algae, and protozoa. The term “microbial” indicates pertaining to, or characteristic of a microorganism.

“Non-pathogenic bacteria” refers to bacteria that under normal conditions do not cause a disease or harmful responses in a healthy host. In some embodiments, non-pathogenic bacteria are commensal bacteria. Examples of non-pathogenic bacteria include, but are not limited to Brevibacillus spp., Bacillus spp., Bacteroides spp., Bifidobacterium spp., Brevibacterium spp., Clostridium spp., Enterococcus spp., Escherichia coli, Lactobacillus spp., Lactococcus spp., Saccharomyces spp., and Staphylococcus spp. Naturally pathogenic bacteria may be genetically engineered to provide reduced or eliminate pathogenicity according to standard methods in the art. Non-pathogenic bacteria may be genetically engineered to enhance or improve desired biological properties, e.g., survivability. Non-pathogenic bacteria and/or yeast may be genetically engineered to provide probiotic properties. Bacteria and/or yeast may be genetically engineered to be non-pathogenic. Without being limited to a particular mechanism of the disclosure, probiotics differ in their ability to produce neurochemicals in the gut of a subject. Non-pathogenic bacteria may be used for probiotic or synbiotic compositions used to treat subjects, while either pathogenic or non-pathogenic bacteria may be used for production of dopamine in a bioreactor or media.

“Commensal microbiota” refers to the population of microbes, including bacteria, that inhabit the body or mucosa and do not typically cause disease or pathology. As used herein, “commensal microbiota” can refer to microbes that produce histamine, including, for example, Bacteroides spp., Clostridium spp., including Clostridium perfringens and Clostridium septicum, Bifidobacterium spp., Enterobacter spp., Escherichia spp., Fusobacterium spp., Klebsiella spp., including Klebsiella aerogenes, Lactobacillus spp., Morganella spp., Proteus spp., or Salmonella spp.

The term “population”, as used herein, refers to a plurality of individual organisms, in the context of this disclosure, the term refers in particular to a collection of organisms of diverse taxonomic affiliation, in particular bacteria.

“Probiotic” is used to refer to live, non-pathogenic microorganisms, e.g., bacteria or fungi which may confer health benefits to a host organism that contains an appropriate amount of the microorganism. In some embodiments, the host organism is a farm production animal, companion animal, aquaculture and/or human. Some species, strains, and/or subtypes of non-pathogenic bacteria are currently recognized as probiotics. Examples of probiotics include, but are not limited to, Candida spp., Debaryomyces spp., Debaryomyces spp., Enterococcus spp., Kluyveromyces spp., Kluyveromyces spp., Saccharomyces spp., Yarrowia spp., Bifidobacteria spp., Escherichia coli, Vagococcus spp., Carnobacterium spp., Melissococcus spp. and Lactobacillus spp., e.g., Candida humilis, Debaryomyces hansenii, Debaryomyces occidentalis, Kluyveromyces lactis, Kluyveromyces lodderae, Kluyveromyces marxianus, Saccharomyces cerevisiae, Saccharomyces boulardii, Yarrowia lipolytica, Bifidobacterium bifidum, Enterococcus faecium, Enterococcus faecalis, Enterococcus hirae, Enterococcus casseliflavus, Enterococcus gallinarum, Escherichia coli strain Nissle, Lactobacillus acidophilus, Lactobacillus bulgaricus, Lactobacillus paracasei, Lactobacillus plantarum, Vagococcus fluvaialis (Dinleyici et al., 2014; U.S. Pat. Nos. 5,589,168; 6,203,797; 6,835,376). A probiotic may also be a variant or a mutant strain of bacterium (Arthur et al., 2012; Cuevas-Ramos et al., 2010; Olier et al., 2012; Nougayrede et al., 2006).

As used herein, the term “synbiotic” means a mixture of probiotic and prebiotics which may confer health benefits to a host organism. The term “prebiotic” as used herein means a cofactor that may improve the survivability of the probiotic microorganisms or induce the growth of activity of beneficial microorganisms such as bacteria or fungi. Therefore, a synbiotic composition is a probiotic composition comprising a probiotic and a cofactor, and, as such, probiotic and synbiotic are used interchangeably herein.

As used here, the term “therapeutically acceptable excipient” means a material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each excipient must be “acceptable” in the sense of being compatible with the other ingredients of the formulation and not injurious to the subject. Some examples of materials which can serve as therapeutically acceptable excipients include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “therapeutically acceptable excipient” or the like are used interchangeably herein.

By “treatment”, “treat,” “prevention,” “prevent” or the like of an adverse condition, infection and/or disease is meant delaying or preventing the onset of such a condition, infection and/or disease, reversing, alleviating, ameliorating, inhibiting, slowing down or stopping the progression, aggravation, or deterioration or severity of a condition associated with such an adverse condition. In one embodiment, at least one symptom of an adverse condition is alleviated by at least 1%, at least 5%, at least 10%, at least 20%, at least 30%, at least 40%, or at least 50%.

The term “sufficient amount of time,” as used herein, refers to the time it takes for a compound, probiotic strain, or the like which is effective for producing some desired effect in at least a sub-population of cells.

The term “E.C.” or “EC” as used herein, refers to the Enzyme Commission number. The E.C. number does not refer to specific enzymes, but to enzyme-catalyzed reactions. Therefore, they represent a group of enzymes that catalyze the same reaction. The E.C. may have up to four numbers which each successive number representing a group of enzymes with a more specific function and/or substrate.

### Histamine

As used herein, the term “histamine” is a small organic molecule C₅H₉N₃ that is a biogenic amine that participates in neural, immune and other general physiological activities. Specifically, histamine is a compound released by cells, primarily mast cells, in response to injury, infection, allergy and/or inflammatory reactions. Diet may also contribute to the amount of histamine present in the body as a reduction in histamine in the diet has been shown to be linked to the reduction of atopic dermatitis symptoms in humans. Histamine is generally formed across all species, both eukaryotic and prokaryotic, by decarboxylation of histidine though a histidine decarboxylase (E.C. 4.1.1.22).

Histamine, through its various receptors has been implicated in a wide range of physiological and behavioral processes. Through their receptors, histamines primarily act as immune mediators and neurotransmitters. The compound generally causes dilation of capillaries, contraction of smooth muscles, and stimulation of gastric acid secretion. Histamines may also affect behavior, such as locomotor activity like scratching, sleep-wake cycles and wakefulness, cognition, and feed intake.

There are currently four known histamine receptors (NCBI MeSH ID: D011968), abbreviated H₁-H₄ or H₁R-H₄R. These receptors are characterized by their binding affinity to various histamine agonists and show some organ and cell specificity. This affinity and specificity modulate the various physiological and behavioral responses to histamine.

H₁ receptors (NCBI MeSH ID: D011969) generally work through the Gαq/11 pathway activating the inositol phosphate/diacylglycerol second messenger system to increase intracellular Ca²⁺. This increase results in smooth muscle contraction, increased vascular permeability, hormone and cytokine release, induces the production of chemokines, prostacyclin and platelet activating factor, and cerebral glyconeogenesis. Hence, almost all immediate hypersensitivity reactions and symptoms observed in the skin, such as erythema, pruritus, and edema, may be elicited through histamine.

H₁ receptors are primarily immune related receptors. They may enhance Th1 and Th2 immune responses by releasing IL-4 and IL-13 to differentiate Th0 cells as well as inhibiting INF-γ and causing Th2 migration to the lungs. Their activation may also lead to B cell proliferation.

H₁ receptors, like H₃ receptors below, have also been implicated in feeding behavior and energy homeostasis. Antipsychotic drugs may act as H₁ antagonists and have been shown to cause significant weight gain in humans. Hence, systemic histamine may affect feeding behavior to such a degree as to cause disease in an organism and returning it to normal levels will return the behaviors to normal. The gut-brain axis consists of bidirectional communication between the central and the enteric nervous system, linking emotional and cognitive centers of the brain with peripheral intestinal functions. Therefore, a change in the histamine level in the gut may work through the gut-brain axis to change these behaviors.

H₁ receptors are also expressed in dermal dendritic cells and keratinocytes in skin tissue. Here H₁ receptors, in addition to differentiation and recruitment of immune cells, increase IL-31 production. This affects pruritus and skin barrier functions in allergic dermatitis. Therefore, H₁ receptors may also induce the various symptoms related with skin diseases, such as atopic dermatitis, and the augmentation of the immune system with histamine may increase the immune response to skin diseases, such as Clostridial dermatitis, to exacerbate the dermatitis. As such, it may be desirable to reduce histamine in a subject to control symptoms.

H₂ receptors (NCBI MeSH ID: D011970), unlike H₁ receptors, are expressed on a wide range of cells and tissues, including B and T cells, dendritic cells, gastricparietal cells, smooth muscle cells, brain, and cardiac tissues. They are coupled to Gas stimulated adenylyl cyclases and can induce airway mucus production, vascular permeability, inotropic and chronotropic effects of heart muscle, relaxation of smooth muscle, and secretion of gastric acid. Impairment of H₂ receptors have been associated with impaired immune functions, gastric acid secretion, and certain cognitive functions, including hippocampal potentiation impairments, which may affect learning and memory, and nociception abnormalities.

H₃ receptors (NCBI MeSH ID: D018100) are expressed exclusively in neurons and coupled to Gαi/o. Here they act as inhibitory auto-receptors and regulate the release of several neurotransmitters in the central and peripheral nervous systems. They are important for homeostatic regulation of energy levels, sleep-wake cycles, cognition, and inflammation. Impaired H₃ receptors and/or antagonists may impair locomotion and effect behavior. This may result in metabolic syndrome characterized by late onset obesity due to hyperphagia and increased leptin and insulin levels. H₃ antagonists have been shown to reverse diet-induced obesity. H₃ receptor antagonists may also be myocardial ischemic arrhythmias, cognition disorders, and insomnia.

Taken together, the stimulation of H₁ and H₃ receptors are at least partially responsible for overall feeding behavior and effect it in such a way that may cause disease when perturbed. When perturbed by a loss of histamine, a subject may alter their normal behavior and exhibit hyperphagia which may result in late onset obesity. Therefore, increasing systemic histamine will decrease feed intake and prevent obesity. However, in healthy subjects the increased histamine may cause undesired weight loss.

H₄ receptors (NCBI MeSH ID: D000074040) are Gα/io coupled and are also expressed on a variety of cells, including immune cells, including eosinophiles, T cells, dendritic cells and mast cells, as well as the spleen, intestinal epithelia, lungs, synovial tissue, central nervous system, sensory neurons, and cancer tissues. H₄ receptors mediates the pro-inflammatory responses of histamine in both an autocrine and paracrine manner. This causes an increased migration of eosinophils, mast cell activation, the expression of pro-inflammatory cytokines and chemokines. H₄ receptor activation, therefore, results in an increase in chemotaxis of mast cells and their accumulation at sites of allergic responses; primes mast cells for allergen-induced activation; and Ca²⁺ dependent mast cell activation. As the recruitment of mast cells may lead to the amplification of an immune response, it may be desirable to reduce the histamine in a subject to prevent chronic inflammation.

H₄ receptors also increase bone marrow-derived basophils following antigen stimulation. This basophil regulation has been associated with allergic dermatitis by inducing chemotaxis of the basophils. Therefore, reducing histamine, such as by providing a probiotic containing a histamine degrading enzyme or organism, may treat and/or prevent dermatitis by lowering the stimulation of the immune cells through H₁ and H₄ receptors.

H₄ receptors are also preferentially expressed in Th2 cells over native T and Th1 cells and may be involved in the pathogenesis of allergy and inflammation. Therefore, reduction of histamine may also prevent the pathogenesis related to chronic allergen exposure and inflammation similar to the treatment of dermatitis.

Taken together, reducing histamine, such as by providing a probiotic containing a histamine degrading enzyme or organism, may treat and/or prevent diseases related to inflammation, such as dermatitis, behavior, and digestion in a subject.

Breakdown of histamine has multiple pathways (KEGG Pathway ID: ko00340) but is typically performed by a class of enzymes belonging to oxidoreductases which act on the CH—NH2 group of the doner compound (E.C. 1.4). More preferably, enzymes belonging to E.C. 1.4.3.22. For example, the amine oxidase (copper containing) (AOC) family of enzymes: AOC1, AOC2, and AOC3, also known in the art as diamine oxidase (DAO), histaminase, histamine oxidase, amiloride-sensitive amine oxidase (copper-containing), amine oxidase copper domain-containing protein, amiloride-binding protein 1 (ABP1), and kidney amine oxidase.

In other organism, such as those lacking an immune system, histamine may still be used in addition to produced. For example, while bacteria may produce histamine, they may also use histamine as a carbon source.

In certain embodiments, the methods described herein result in reduction of the microbial production of histamine in a subject such that the negative effects of histamine are eliminated or reduced. In certain embodiments, the production of histamine is reduced by about 10%, about 20%, about 30%, about 40%, about 50%, about 60%, about 70%, about 80%, or about 90% as compared to the microbial production of histamine in a subject prior to treatment.

### Subjects

The probiotic or synbiotic compositions and/or histamine degrading enzymes and methods of use described herein are particularly suitable for use with farm production animals, companion animals, aquaculture, and humans.

Farm production animals include for example, poultry, turkeys, and any avian species. Farm production animals further include ruminants and non-ruminants, including cows, pigs, goats, sheep, and the like. Companion animals include for example, dogs, cats, and horses. Aquaculture includes all species.

The probiotic or synbiotic compositions and/or histamine degrading enzymes and methods of use described herein are particularly suitable for use in avian species. All birds are part of the Class Ayes (Phylum Chordata and Subphylum Vertebrata). Examples of suitable avian species that may be treated with the probiotic compositions described herein include, but are not limited to, chickens, ducks, geese, turkeys, guinea fowl, ostriches, pigeons, quails, and pheasants. Examples of avian farm production animals most often include chickens and turkeys.

Probiotic Compositions and/or Histamine Degrading Enzymes

The probiotic or synbiotic compositions may comprise one or more live bacteria and/or yeast, extracts or purified protein obtained thereof, and/or synthetic enzymes which may break down histamine if it is present in a subject. The probiotic compositions may include a strain(s) administered as a probiotic bacterial and/or yeast strain that utilizes histamine for its physiology. In one embodiment, the probiotic compositions may include a strain(s) administered as a probiotic bacterial strain that utilizes histamine for its physiology (i.e. consumes or degrades histamine). The probiotic composition could alternatively administer a spore which then germinate into a histamine-degrader in the gut of the subject. In a further embodiment, the probiotic bacterial strain may further be modified to knock down or knock out histidine decarboxylase to prevent additional histamine from being produced by the probiotic strain.

In a further embodiment, the probiotic compositions may be provided as a mash in which the probiotic is incorporated and processed to release the enzymes contained within that can break down histamine in the gut. Exemplary enzymes include amine oxidase (copper containing). Examples of use of these enzymes in food preparations have been shown, such as the preparation of tuna soup containing very high levels of histamine, wherein the enzyme amine oxidase (copper containing) is shown to reduce histamine during the preparation of the soup. Without being limited to a particular mechanism of action, the activity of the amine oxidase (copper containing) was destroyed during the preparation in a final step of boiling, so no histamine degradation was ever proposed to occur following food consumption. Analogous studies employ such enzymes only during preparation of food and then destroy the activity prior to consumption. See Naila et al. Prediction of the amount and rate of histamine degradation by diamineoxidase (DAO). Food Chemistry 135:2650-2660, 2012.

In other embodiments, the compositions may comprise of enzymes belonging to E.C. 1.4.3, such as amine oxidase (copper containing), facilitate the reaction RCH₂NH₂ (amine)+H₂O (water) to RCHO (aldehyde)+NH₃ (ammonia)+H₂O₂ (peroxide). Specifically, amine oxidase (copper containing) converts histamine (amine) into imidazole acetaldehyde (aldehyde), ammonia and peroxide. Many genera, including but not limited to bacteria, fungi, and plants, have been reported to possess this activity. Prominent examples of bacteria include strains of Staphylococcus including S. xylosus, S. cronusus; strains of Brevibacterium spp. including B. linens; Arthrobacter crystallopoietes; some Lactobacilli spp. as well as Vergibacillus spp., Pseudomonas spp., and Escherichia spp. The efficacy of these enzymes is maximized under neutral to alkaline conditions and oxygen must be available. It has been reported that amine oxidase (copper containing) can be directly added to certain fermented foods to achieve a decrease in histamine levels.

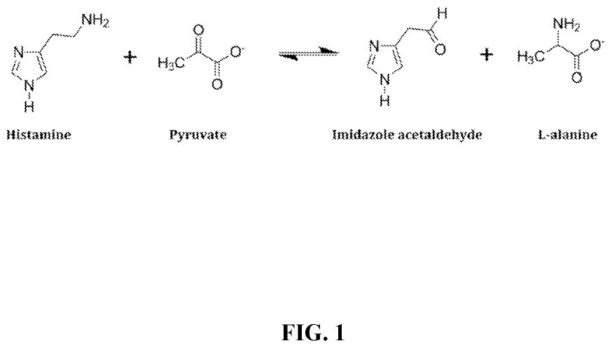

In other embodiments, the compositions may contain enzymes which may degrade histamine by a dehydrogenase pathway. In this variant, histamine once again is utilized to generate imidazole acetaldehyde and ammonia. However, in contrast to oxidase pathways, dehydrogenase pathways frequently involve several steps including a transamination step and dehydrogenation step. This reaction also does not generate hydrogen peroxide. Consider the pathway recently described for Pseudomonas putida. In this pathway, several enzymes work in a concerted process. In particular HinC, a histamine-pyruvate aminotransferase (E.C. 2.6.1.58), transfers an amine from histamine to pyruvate and in the process generates alanine (FIG. 1).

Imidazole acetaldehyde can be further processed by an aldehyde dehydrogenase (E.C. 1.2.1) enzyme like HinD to yield imidazole acetic acid. Pyruvate can also be regenerated from L-alanine, a process which liberates ammonia. Species notable for histamine degradation by dehydrogenase pathways include Rhizobium, Nocardioides simplex and Natrinema gari. Because this variant relies on transamination, it is conceivable that an abundance of a preferred amine bearing substrates may decrease the degradation of histamine whereas an abundance of pyruvate might enhance histamine degradation.

In certain embodiments, the histamine degrading enzymes comprise cell fragments that still retain membrane-bound histamine degrading enzymes. Such cell fragments can be produced by known methodologies. For example, one could use a French Press—the application of high pressure to force a suspension of microorganisms through a small orifice. At such high pressures, the integrity of the cells is disrupted and the cell bursts open. Another example would be the use of sonication which involves the generation of ultrasonic vibration that when applied in a solution containing microorganisms, either live or dead, results in the fragmentation of the cells. In a third example, the use of a bead rupturing technology can be employed wherein small diameter beads (millimeters or less) known as lysing beads are added to a solution of live or dead microbes. The tube containing the lysing beads and microbes is then uniformly shaken at high-speed resulting in homogenization of the cell. The result from all three of these examples is the production of cellular fragments comprising either the cell wall, membrane, or internal membranes. In certain embodiments, these cellular fragments still possess intact and functional histamine degradative enzymes. This mixture of cell fragment/membranes containing histamine degradative enzymes can then be added to a food or water source. In this way, microorganisms that may not qualify as European Food Safety Authority (EFSA) approved can be utilized in animals since no live cells are being administered. The results of the administration of this mixture would be a decrease in gut or systemic levels of histamine.

In an embodiment, probiotics may be grown in large fermenters and then pelleted, dried, and ground down. This processing destroys the viability of the organism yet preserves the activity of their enzymes to then allow the mash to be incorporated into the feed. Such methods must be done in accordance with accepted methods of preparation bacterial or fungal products for use in feeds.

In an embodiment, probiotics may be grown in large fermenters and then simply heat-inactivated with no further processing and then incorporated into the feed.

In an embodiment, the probiotics may not have a histidine decarboxylase (E.C. 4.1.1.22) enzyme or have been modified to knock down or knock out the function of histidine decarboxylase to prevent the probiotic from making histamine.

In yet a still further embodiment, the enzymes, such as in the form of purified enzyme (or one or more of the bacterial or yeast species listed herein) can be administered or incorporated into feed. Exemplary enzymes include amine oxidase (copper containing). In embodiments, enzymes can be coated to protect their activity during transit through the gut until they reach specific section of gut where the need for histamine degradation exists.

In further embodiments, for use in animal feeds, enzymes can be added “neat” but also can be coated to protect their activity. There are a number of coating strategies that are currently used to protect enzymes in feed. By way of nonlimiting example, different types of granulation or nanoparticle techniques may be used to coat the enzyme including providing a core or inner matrix to which the enzyme may be attached and then using one or more coatings to protect the enzyme from the heat, pressure, and moisture generated during feed manufacturing and then to allow delivery of a sufficient amount of the enzyme to the subject. Further see Hua S. (Advances in Oral Drug Delivery for Reginal Targeting in the Gastrointestinal Tract—Influence of Physiological, Pathophysiological and Pharmaceutical Factors, Front. Pharmacol, 2020, 11:524, herein incorporated by reference in its entirety), Fenster, K., et al. (The Production and Delivery of Probiotics: A Review of a Practical Approach, Microorganisms 2019, 7, 83, herein incorporated by reference in its entirety), and Govender, M., et al. (AAPS PharmSciTech, 2014, 15, 29, herein incorporated by reference in its entirety) for delivering compounds to the gastrointestinal tract in general.

Histamine may be metabolized through at least four known mechanisms (as described in https://www.histaminintoleranz.ch/en/histaminosis_histaminemetabolism.html), including (1) Oxidative deamination through amine oxidase (copper containing), also known as DAO or histaminase, according to the following chemical equation: Histamine+H2O+O2=>(imidazole-4-yl)acetaldehyde+NH3+H2O2; (2) Cyclical methylation through histamine N-methyltransferase (HNMT; E.C. 2.1.1.8), wherein the resulting product is N-methylhistamine (NMH); (3) Acetylation into acetylhistamine, which is the degradation pathway most important in microbial degradation by enzymes in E.C. 2.3.1; and (4) Hydroxylase into hydantoin propionic acid, wherein the enzymes can be present in microorganisms.

In embodiments, the composition includes at least one bacterial probiotic strain capable of degrading histamine. In certain embodiments, the compositions include at least one Brevibacillus spp. in the probiotic compositions. Brevibacillus spp. are Gram-positive, aerobic, endospore-forming bacterium. The Brevibacillus spp. strains can also be given as a probiotic (non-spore) or as a spore. In a preferred embodiment and without limitation to use of other bacteria and fungi described herein, the probiotic compositions described herein include a Brevibacillus spp. Without being limited to a particular mechanism of action, the Brevibacillus spp. degrades histamine. Exemplary Brevibacillus include, but are not limited to, Brevibacillus borstelensis, Brevibacillus brevis, Brevibacillus agri, Brevibacillus antibioticus, Brevibacillus aydinogluensis, Brevibacillus centrosporus, Brevibacillus chloshinensis, Brevibacillus composti, Brevibacillus fluminis, Brevibacillus formosus, Brevibacillus fortis, Brevibacillus fulvus, Brevibacillus gelatini, Brevibacillus ginsengisoli, Brevibacillus halotolerans, Brevibacillus invocatus, Brevibacillus laterosporus, Brevibacillus levickii, Brevibacillus limnophilus, Brevibacillus marinus, Brevibacillus massiliensis, Brevibacillus migulae, Brevibacillus nitrificans, Brevibacillus panachumi, Brevibacillus parabrevis, Brevibacillus porter, Brevibacillus reuszeri, Brevibacillus schisleri, Brevibacillus sediminis, Brevibacillus texasporus, and Brevibacillus thermoruber.

In an embodiment the Brevibacillus spp. is Brevibacillus borstelensis, Brevibacillus brevis, and/or Brevibacillus parabrevis.

In certain embodiments, probiotic compositions of the present disclosure include at least one Bacillus spp. Exemplary Bacillus include, but are not limited to Bacillus subtilis, Bacillus cereus, Bacillus anthracis, Bacillus thuringiensis, Bacillus licheniformis, Bacillus megaterium, Bacillus pumilus, Bacillus coagulans, Bacillus stearothermophilus, Bacillus circulans, Bacillus brevis, Bacillus amyloliquefaciens, Bacillus polymyxa, Bacillus mycoides, Bacillus sphaericus, Bacillus clausii, Bacillus firmus and Bacillus flexus.

In certain embodiments, the Bacillus spp. is Bacillus flexus and/or Bacillus subtilis.

In some embodiments, the probiotic compositions include a therapeutically effective amount of at least one live, non-pathogenic probiotic Brevibacillus or Bacillus strain, such as those listed above. In some embodiments the bacterial strains utilize histamine for its own survival and growth, thereby overcoming the excess histamine production by certain commensal microbiota.

In an embodiment, a therapeutically effective amount of the bacterial probiotic strain(s) is from about 10⁴ CFU to about 10¹⁴ CFU. In another embedment, a therapeutically effective amount of the histamine degrading enzyme to be used in combination with a bacterial probiotic strain(s) is from about 10¹ histamine degrading units (HDU) to about 10¹⁰ HDU. In another embodiment, a therapeutically effect amount of the composition may be from about 10⁴ CFU to about 10⁴ CFU of a bacterial probiotic strain and from about 10¹ HDU to about 10¹⁰ HDU histamine degrading enzyme. As used herein, a HDU is the number of molecules of histamine that may be degraded.

Additional strains can be combined with the bacterial probiotic strain for inclusion in the probiotic compositions or to extract enzyme. The various strains can also be given as a probiotic (non-spore) or as a spore. Exemplary additional bacterium include: Arthrobacter spp., including for example A. crystallopoietes; Bacillus spp., including for example B. subtilis, B. coagulans, B. lichenformis, and B. amyloliquefaciens; Brevibacterium spp., including for example B. linens; Micrococcus spp.; Rhizobium spp.; Enterococcus spp., including for example E. cecorum; Escherichia spp., including for example E. coli; Lactobacillus spp., including for example L. sakei, L. plantarum, and L. crispatus; Staphylococcus spp., including for example S. xylosus, S. caronusus; Agrobacterium spp., including for example A. tumefaciens; Vergibacillus spp., including for example V. halodenitrificans; Pseudomonas spp., including for example P. putida or P. aeruginosa; Candida spp., including for example C. krusei; Nocardioides spp., including for example N. simplex; Rummeliibacillus spp., including for example Rummeliibacillus stabekisii; Natrinema spp., including for example Natrinema gari; Debaryomyces spp. (yeast), including for example D. hansenii (fungi); Saccharomyces spp. (yeast); Yarrowia spp., including for example E. lipolytica; Aspergillus spp., including for example Aspergillus oryzae or Aspergillus niger; Penicillium spp., including for example Penicillium italicum; Pinchia spp., including for example Pichina pastoris; and Gibberella spp., including for example Gibberella fujikuroi.

The bacterial probiotic compositions and/or histamine degrading enzymes may be administered to reach the gut as a lyophilized powder or in a tablet form. The lyophilized powder may be added to a liquid such as, but not limited to, water or food for ingestion. The tablet may be a chewable tablet. The bacterial probiotic may be administered live or heat inactivated dead cells, and in whole or in part. The parts of the probiotic may include cellular components, such as, but not limited to, the DNA or protein which are capable of rendering their beneficial effects. The bacterial probiotic compositions may be administered in any pharmaceutically acceptable formulation such as, but not limited to, a tablet or as part of a composition comprising the substrate and a pharmaceutically acceptable carrier.

Tablets and capsules for administration to the gut may be in unit dose form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tableting lubricant, for example magnesium stearate, talc, polyethylene glycol or silica; disintegrates for example potato starch, or acceptable wetting agents such as sodium lauryl sulphate. The tablets may be coated according to methods well known in normal pharmaceutical practice. Preferred coatings and encapsulation agents include pH-sensitive biocompatible polymers. By way of non-limiting example, these polymers may include poly-lactic acid, poly(lactic-co-glycolic acid), and chitosan. Other coatings, such as but not limited to hydroxypropyl methylcellulose phthalate or carboxymethyl high amylose starch, may also be used to enhance the delivery of probiotics to the gut. Alternatively, the coating may take advantage of the bacteria found in the gut and comprise a polymer that show degradation specificity for different regions of the gut. While biodegradable coatings may be used, they are not preferred because they can suffer from premature drug release or bursting due to their hydrophilicity and solubility in regions various regions of the gut, for example, in the upper gastrointestinal tract. The coatings may also contain more than a single compound or layer. An example of a multilayer coating may include a first protective coating which becomes degraded in the stomach, a second layer of a pH-dependent polymer coating which becomes degraded in the small intestine, and a third coating which the microorganism commensal to the colon may break down to release the probiotic to the colon. For examples, see Hua S. (2020), Fenster, K. (2019), and Govender, M. (2014).

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, Sorbian monooleate, polysorbate 80, or acacia; non-aqueous vehicles (which may include edible oils), for example almond oil, fractionated coconut oil, oily esters such as glycerin, propylene glycol, or ethyl alcohol; preservatives, for example methyl or propyl p-hydroxybenzoate or sorbic acid, and if desired conventional flavoring or coloring agents.

Topical compositions may be administered as live or heat inactivated dead cells, and in whole or in part. The parts of the probiotic may include cellular components, such as, but not limited to, the DNA or protein which are capable of rendering their beneficial effects. The probiotic compositions may be administered in any pharmaceutically acceptable formulation such as may be formulated as a lotion, shake lotion, cream, ointment, gel, foam, powder, solid, past, or tincture. In a further environment, the topical composition may further comprise a bandage, dressing, or combinations thereof.

In a further embodiment, a bandage or dressing is provided comprising the topical probiotic compositions described above. In yet further embodiments, a bandage or dressing is provided comprising the topical probiotic compositions described above, glycerol, and any combination thereof. In various aspects, a bandage or dressing is provided the major constituents of which includes a matrix and a topical probiotic composition. In various aspects, a bandage or dressing is provided the major constituents of which includes a matrix and a probiotic. In various aspects, a bandage or dressing is provided the major constituents of which includes a matrix and glycerol.

Methods of Prevention and/or Treatment Using Probiotic Compositions and/or Histamine Degrading Enzymes

Various conditions are suitable for prevention and/or treatment using the probiotic compositions and/or histamine degrading enzymes. In an embodiment, avian species are particularly in need of prevention and/or treatment using the probiotic compositions and/or histamine degrading enzymes for reducing gut inflammation, reducing incidence of Clostridial dermatitis, decreasing incidence and pathogenesis of potential pathogens for which histamine is a virulence factor, improving and stabilizing gut health, increasing feed efficiency, and/or improving behavior through decrease in stress-related histamine-mediated pathways.

In an embodiment, porcine species are particularly in need of prevention and/or treatment using the probiotic compositions and/or histamine degrading enzymes for reducing gut inflammation, decreasing incidence and pathogenesis of potential pathogens for which histamine is a virulence factor, improving and stabilizing gut health, increasing feed efficiency, and improving behavior through decrease in stress-related histamine-mediated pathways.

In an embodiment, ruminant species are particularly in need of prevention and/or treatment using the probiotic compositions and/or histamine degrading enzymes for reducing gut inflammation, decreasing incidence and pathogenesis of potential pathogens for which histamine is a virulence factor, improving and stabilizing gut health, increasing feed efficiency, and improving behavior through decrease in stress-related histamine-mediated pathways.

In an embodiment, aquaculture species are particularly in need of prevention and/or treatment using the probiotic compositions and/or histamine degrading enzymes for reducing gut inflammation, decreasing incidence and pathogenesis of potential pathogens for which histamine is a virulence factor, improving and stabilizing gut health, controlling and treating a variety of fish-related neoplastic diseases increasing feed efficiency, and improving behavior through decrease in stress-related histamine-mediated pathways.

In an embodiment, human species are particularly in need of prevention and/or treatment using the probiotic compositions and/or histamine degrading enzymes for reducing gut inflammation, managing and treating inflammatory and autoimmune disorders as the conditions can be influenced by the body's capacity for histamine degradation, treating gastrointestinal ailments including diffuse stomach ache, colic, flatulence, and diarrhea which are leading symptoms of histamine intolerance, controlling and treating a variety of neoplastic diseases such as Crohn disease and ulcerative colitis as well as other gastrointestinal conditions such as allergic enteropathy and food allergy which have been shown to involve elevated histamine concentrations and diminished histamine degradative enzymes in the gut such as diamine oxidase, use of histamine degradation strategies in the gut to manage airway impairments such as asthma and rhinorrhea as well as some sexual dysfunctions like dysmenorrhea, improving behavior through decrease in stress-related histamine-mediated pathways.

In still further embodiments, methods of preventing and/or treating clostridial dermatitis in a subject with a probiotic composition and/or histamine degrading enzymes are herein provided. Further, methods of preventing and/or treating clostridial enteric disease with a probiotic composition and/or histamine degrading enzymes are provided. Further, methods of preventing and/or treating necrotizing enterocolitis with a probiotic composition and/or histamine degrading enzymes are provided. Still further, method of preventing and/or treating gut inflammation with a probiotic composition and/or histamine degrading enzymes are provided.

Further, method of preventing and/or treating all aspects of gut-related health in a subject, namely a human subject is provided. As Clostridium spp. and Escherichia spp. produce histamine there is a direct connection and relevance to gut-related diseases involving microbial-produced histamine.

The various infections, diseases and conditions for which prophylaxis and treatment according to the methods described herein are suited, may include a commensal microbiota, including Clostridium spp., causing an increase in histamine production in the subject. Exemplary Clostridium spp. include Clostridium perfringens and Clostridium septicum, including strains having Genbank accession no. KX674025, KX674031 and KX674026. Beneficially, the methods of preventing and/or treating using the probiotic compositions control histamine by providing a probiotic strain that degrades histamine, providing the subjects' immune system time and ability to respond.

The methods include delivering a probiotic composition and/or histamine degrading enzymes to the subject. The delivering of the compositions and/or histamine degrading enzymes can be provided in any conventional manner of dosing. For example, the probiotic composition and/or histamine degrading enzymes can be provided in a feed source, such as pellets. In certain embodiments, the probiotic composition and/or histamine degrading enzymes can be provided in a water source, including directly to the water in the case of aquaculture species.

The methods further include controlling histamine production. In certain embodiments, the controlling of histamine is a reduction in histamine production within the subject, including histamine produced by the subject, histamine ingested by the subject, and/or histamine produced by a microbe within the subject. In certain embodiments, the controlling of histamine is a reduction in histamine production by a microbe within the subject, including commensal and non-commensal microbes. Histamine producing microbes can include, but are not limited to, Clostridium spp., Salmonella spp., Escherichia spp., Proteus spp., Morganella spp., Enterobacter spp., Klebsiella spp., and Lactobacillus spp. In an embodiment, the histamine producing microbe is Clostridium perfringens. The delivering of the probiotic compositions and/or histamine degrading enzymes can include administering to a subject with an active microbial infection, or the administering to a subject as prophylaxis.

Without being limited to a particular mechanism of action, the method for prevention and/or treatment of a subject for clostridial dermatitis, clostridial enteric disease, necrotizing enterocolitis, and/or gut inflammation with a probiotic composition and/or histamine degrading enzyme that beneficially controls histamine production caused by a commensal microbiota, including for example Clostridium perfringens which have been shown to result in excess histamine production.

As one skilled in the art recognizes, there is a biochemical signaling in the gut-brain axis joining the microbiota, the alimentary tract (including the gastrointestinal tract) and the central nervous system. The gut-brain axis includes the microbiota in the alimentary tract, central nervous system, neuroendocrine and neuroimmune systems (e.g. hypothalamic—pituitary—adrenal axis), sympathetic and parasympathetic arms of the autonomic nervous system, and the gut microbiota. Beneficially, the methods of prophylaxis and treatment are suitable for adjuvant treatment of various pathologies of the gut. Exemplary gut inflammation can include various gastrointestinal conditions, including for example, ulcers, namely gastric ulcers, diarrhea, inflammatory bowel disease (IBD) and associated symptoms and conditions, feeding conditions causing behavioral abnormalities, enterocolitis-type inflammation, and the like. As described herein, a consequence of the dysregulated production of histamine is the effect on neuroimmune interactions driving inflammation and behavior.

The neuroimmune system includes the structures and processes involving the biochemical and electrophysiological interactions between the nervous system and immune system. These two systems are tightly integrated and have both local and systemic reflexes to restore homeostasis in response to injury and/or infection. The neuroimmune system is comprised primarily of glial cells and mast cells. As discussed above, histamine receptors, especially H₄, are expressed on mast cells and drive their activation. They key role of mast cells in inflammation and in the disruption of the blood-brain barrier may be achieved through the activation of the mast cells through these receptors. Mast cells also seem to participate in neuroinflammation both directly and through microglia stimulation. These two systems also communicate using a system of broad, common cytokines, growth factors, and neuropeptides, allowing for bidirectional communication. This cross talk permits the amplification of maladaptive inflammatory feedforward loops. Hence, the activation of mast cells through histamine receptors may contribute to the pathogenesis of such conditions such as headaches, autism, chronic fatigue syndrome, allergy, asthma, chronic coughing, autoimmunity, itch, and pain. As the gut-brain axis connects the gut to the brain, this is also linked to the neuroimmune system through the communication between the immune and nervous systems. Therefore, histamine production in the gut may influence behavioral changes through the gut-brain axis and mediated by mast cells. Accordingly, the methods described herein able to control histamine production by probiotic compositions and/or histamine degrading enzymes beneficially control neuroimmune events, such as headaches, autism, chronic fatigue syndrome, allergy, autoimmunity, itch, and pain, in the subject.

The methods include administering to the subject a therapeutically effective amount of at least one bacterial probiotic strain and/or histamine degrading enzyme. In an aspect, a therapeutically effective amount of the bacterial probiotic strain(s) in the composition includes from about 10⁴ CFU to about 10¹⁴ CFU, from about 10⁴ CFU to about 10¹² CFU, from about 10⁵ CFU to about 10¹¹ CFU, or from about 10⁵ CFU to about 10¹⁰ CFU. In another aspect, a therapeutically effective amount of the histamine degrading enzymes in the compositions include from about 10¹ to about 10¹⁰, from about 10³ HDU to about 10¹⁰ HDU, from about 10⁴ HDU to about 10⁸ HDU, or from about 10⁵ HDU to about 10⁷ HDU. A composition may include a therapeutically effective amount of both the probiotic and the enzyme.

In an embodiment, the method of administering is by oral administration. Oral administration can include various dosage forms as one skilled in the art will ascertain, including for example, tablets, capsules, aqueous or oily suspensions, solutions, emulsions, syrups or elixirs, dry products for reconstitution with water or other suitable vehicle before use.

Tablets and capsules for oral administration may be in unit dose form, and may contain conventional excipients such as binding agents, for example syrup, acacia, gelatin, sorbitol, or polyvinyl-pyrrolidone; fillers for example lactose, sugar, maize-starch, calcium phosphate, sorbitol or glycine; tableting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, polysorbate 80, or acacia; non-aqueous vehicles (which may include edible oils), for example almond oil, fractionated coconut oil, oily esters such as glycerin, propylene glycol, or ethyl alcohol; preservatives, for example methyl or propyl p-hydroxybenzoate or sorbic acid, and if desired conventional flavoring or coloring agents. Tablets and capsules may be formulated as a time release tablet or capsule to target different organs along the alimentary track. Similarly, the use of encapsulated and/or coated preparations can be employed, such as for the histamine degrading enzyme preparations.

In an embodiment, the method of administering is by topical administration. Topical administration can include various dosage forms as one skilled in the art will ascertain, including for example, lotions, creams, shake lotion, cream, ointment, gel, foam, powder, solid, paste, or tincture.

In another embodiment, the method of administering is by a bandage or dressing comprising the topical compositions described above. In a further embodiment, a bandage or dressing is provided comprising the topical probiotic compositions described above, glycerol, and any combination thereof. In various aspects, a bandage or dressing is provided the major constituents of which includes a matrix and a topical probiotic composition. In various aspects, a bandage or dressing is provided the major constituents of which includes a matrix and a probiotic. In various aspects, a bandage or dressing is provided the major constituents of which includes a matrix and glycerol.

All publications, patent applications, issued patents, and other documents referred to in this specification are indicative of the level of ordinary skill in the art to which this disclosure pertains and are herein incorporated by reference to the same extent as if each individual publication or patent application was specifically and individually indicated as incorporated by reference. Definitions that are contained in text incorporated by reference are excluded to the extent that they contradict definitions in this disclosure.

The following numbered embodiments also form part of the disclosure:

1. A method of controlling histamine production in a subject comprising: administering a probiotic composition comprising a Brevibacillus spp. and/or Bacillus spp. and optionally a histamine degrading enzyme to the subject; and controlling histamine production within the subject.

2. The method of embodiment 1, wherein the Brevibacillus spp. is Brevibacillus borstelensis, Brevibacillus brevis, and/or Brevibacillus parabrevis.

3. The method of embodiment 1 or 2, wherein the Bacillus spp. is Bacillus flexus and/or Bacillus subtilis.

4. The method of any one of embodiments 1-3, wherein the subject is a chicken, duck, goose, turkey, guinea fowl, ostrich, pigeon, quail, and pheasant.

5. The method of any one of embodiments 1-4, wherein the subject is a companion animal, farm animal, or aquaculture species.

6. The method of any one of embodiments 1-5, wherein the subject is a human.

7. The method of claim 1, wherein the controlling of histamine is a reduction in the microbial production of histamine.

8. The method of any one of embodiments 1-7, wherein the histamine-degrading enzyme is amine oxidase (copper containing) and optionally wherein the enzyme is a purified enzyme.

9. The method of any one of embodiments 1-8, wherein the Brevibacillus spp. and/or Bacillus spp. is a live microorganism, a dead and/or inactivated microorganism, a spore that is germinated into a histamine-degrader in the subject, or a mash incorporating a microorganism therein.

10. The method of any one of embodiments 1-9, wherein the probiotic composition and optional histamine-degrading enzyme is neat, coated or encapsulated and administered in a feed or water source.

11. A method of preventing and/or treating an inflammatory disease and/or immunological condition mediated by histamine in a subject, comprising: administering a probiotic composition comprising a Brevibacillus spp. and/or Bacillus spp. and optionally a histamine degrading enzyme to the subject; and controlling histamine production within the subject.

12. The method of embodiment 11, wherein the inflammatory disease and/or immunological condition is clostridial dermatitis, clostridial enteric disease, gut inflammation, necrotizing enterocolitis, neoplastic disease, and/or bacterial infection.

13. The method of embodiment 11 or 12, wherein the Brevibacillus spp. is Brevibacillus borstelensis, Brevibacillus brevis, and/or Brevibacillus parabrevis.

14. The method of any one of embodiments 11-13, wherein the Bacillus spp. is Bacillus flexus and/or Bacillus subtilis.

15. The method of any one of embodiments 11-14, wherein the probiotic composition comprises from about 10⁴ CFU to about 10¹⁴ CFU of the Brevibacillus spp. and/or Bacillus spp.

16. The method of any one of embodiments 11-15, wherein the administration is oral or topical.

17. The method of any one of embodiments 11-16, wherein the subject is a pig, chicken, duck, goose, turkey, guinea fowl, ostrich, pigeon, quail, and pheasant.

18. The method of any one of embodiments 11-16, wherein the subject is a companion animal, farm animal, or aquaculture species.

19. The method of any one of embodiments 11-16, wherein the subject is a human.

20. The method of any one of embodiments 11-19, wherein the controlling of histamine is a reduction in the microbial production of histamine.

21. The method of any one of embodiments 11-20, wherein the histamine-degrading enzyme is amine oxidase (copper containing), and optionally wherein the enzyme is a purified enzyme.

22. The method of any one of embodiments 11-21, wherein the Brevibacillus spp. and/or Bacillus spp. is a live microorganism, a dead and/or inactivated microorganism, a spore that is germinated into a histamine-degrader in the subject, or a mash incorporating a microorganism therein.

23. The method of any one of embodiments 11-22, wherein the probiotic composition and optional histamine-degrading enzyme is neat, coated or encapsulated and administered in a feed or water source.

24. A probiotic composition, comprising: a therapeutically effective amount of a Brevibacillus spp. and/or Bacillus spp.; and a therapeutically acceptable excipient, additional functional ingredient, and/or a food/feed component.

25. The composition of embodiment 24, wherein the Brevibacillus spp. is Brevibacillus borstelensis, Brevibacillus brevis, and/or Brevibacillus parabrevis and the Bacillus spp. is Bacillus flexus and/or Bacillus subtilis.

26. The composition of embodiment 24 or 25, wherein therapeutically effective amount of the Brevibacillus spp. and/or Bacillus spp. is from about 10⁴ CFU to about 10¹⁴ CFU.

27. The composition of any one of embodiments 24-26, wherein the additional functional ingredient is a histamine-degrading enzyme.

28. The composition of any one of embodiments 24-27, wherein the histamine-degrading enzyme is histamine oxidase and/or diamine oxidase, and optionally wherein the enzyme is a purified enzyme.

29. The composition of any one of embodiments 24-28, wherein the composition is an oral dosage in liquid or solid form.

30. The composition of any one of embodiments 24-29, wherein the composition is a time release tablet or capsule.

The present disclosure is further illustrated by the following examples, which should not be considered as limiting in any way.

### EXAMPLES

### Example 1

To determine whether the herein disclosed Brevibacillus borstelensis contains known histamine degrading genes, sequence comparisons were carried out against nonredundant protein databases performed using the BLASTP algorithm with the default parameters (Altschul, S. F. et al. Gapped BLAST and PSI-BLAST: a new generation of protein database search programs, Nucleic Acids Res., 25, 3389-3402 1997). BLASTP results providing amino acid identities of greater than about 50%-60% amino acid identity were considered to indicate a positive hit. In principle, identifying sequence similarity with known Pseudomonas putida proteins of defined function in the database provides a means to identify Brevibacillus proteins with a similar function.

Specifically, BLASTP was used to search highly conserved, known Pseudomonas putida histamine degradation genes for homologs in the Brevibacillus genomes currently available at NCBI. Using the NCBI nonredundant protein sequence database, the search results were filtered to only include sequences from the genus Brevibacillus.

The BLASTP search yielded either no hits or no statistically significant positive hits. For example, in one case the search yielded three statistically insignificant hits corresponding to genes found in strains that are known to be capable of histamine degradation: HinF (AWA45229.1), HinI (AWA45233.1), and HinL (AWA45230.1). These search results were considered statistically insignificant because they indicated less than 35% amino acid identity to the Pseudomonas query sequences. Based on the lack of identified sequence similarity (less than 35% amino acid identity) in the BLASTP search, it was concluded that the Brevibacterium disclosed herein does not contain known histamine degrading genes. The analyses performed show that the Brevibacillus genomes in NCBI do not have any clear or convincing homologs of the Pseudomonas putida histamine degradation genes.

### Example 2

For a microorganism to be safe to administer to a subject, they must be non-pathogenic. One such location in which to identify non-pathogenic strains would be naturally occurring bacteria of the gut. This may be accomplished by feeding a subject a histamine-enriched diet that would force the subject's microbiota to enrich for those gut bacteria that could utilize and degrade the exogenous histamine fed as part of the diet. This method differs from a more traditional in vitro screening approach where a whole bank of bacteria is screened for potential histamine degrading activity or through a bioinformatic approach (as described in Example 1).

The aim of this trial was to have the subject, the chickens, do the part of the work in identifying chicken-derived bacteria that could be used as histamine-degrading probiotics. Gram-positive bacteria were targeted as they would include spore formers and having spore forming probiotics is of great advantage to the probiotic industry, as spores are readily incorporated into the manufacturing process due to their ability to survive elevated temperatures commonly used in the production of foods.

### Histamine Feed Trial Experiments and Methodology:

One day old, unsexed, Cobb chickens were randomly divided into two separate groups. Each group was fed the same basal chicken diet supplemented or not with histamine as follows:
1. Group 1 (18 chickens)—Low Histamine: 5 mg of histamine dihydrochloride (Arcos, CAS #56-92-8) was added to 100 g of granular (crumbles) feed
2. Group 2 (18 chickens)—High Histamine: 15 mg of histamine dihydrochloride was added to 100 g of granular (crumbles) feed

All chicken feed was prepared at the Iowa State University feed mill. This amount of histamine was selected to not harm the animals. The histamine was added in as a powder and mixed with the granular chicken feed to obtain a homogeneous mixture. Histamine concentration was verified by measuring on an ultra-high-performance liquid chromatography (UHPLC) instrument as per the description below. During the feeding trial, all chickens were fed, and watered ad libitum and appropriate enrichment items were placed in the pens.

At approximately 3 and 5 weeks of age equal numbers of chickens from each group were euthanized via CO₂ for 10 minutes with cervical dislocation as a secondary method. After death was confirmed, the bifurcated cecum and ileal and colonic segments, were removed, cut open and the contents were obtained and combined into a single individual tube for each chicken. Additionally, the inner mucosal layer of each of the tissues was collected following scraping of the mucosal side of the tissue using the cutting edge of a scissor and added to the individual tube containing the intestinal contents specific for that particular chicken.

### 1. Culturing Technique to Identify Presence of Histamine-Degrading Bacteria:

Following harvest of the intestinal contents and the corresponding tissue mucosal scrapings, the intestinal samples were placed in Sporulation both medium (HiMedia catalog #M1018-500G) that was supplemented with 10 mM histamine dihydrochloride. Cultures were well-mixed at high speed on a vortex to dislodge adherent bacteria contained within the mucosal scrapings and break up bacterial clumps. Duplicate cultures were setup for each chicken biological sample. The first set of duplicate tubes were placed in a 70° C. water bath for 20 minutes with intermittent mixing. Following removal from the water bath the tubes were briefly vortexed and placed into a 42° C. incubator for up to 8 days. The second set of duplicate tubes were directly placed into a 42° C. incubator for up to 8 days. At the end of the 8 day period, the second set of duplicate tubes was then subjected to immersion in a 70° C. water bath for 20 minutes with intermittent mixing. Heating to 70° C. in a water bath for 20 minutes is a well-accepted procedure that has been in use for decades to preferentially select for bacterial spores as only bacterial spores, and not vegetative cells, will survive the prolonged heating.

At specified intervals during the 8 day period, samples were removed and assayed for histamine content and compared against a non-bacterial containing histamine medium control. A decrease in histamine content as compared to the non-bacterial containing control indicated the presence of bacterial histamine degraders. Tubes that were found to have greater than 20% histamine degradation were then plated onto Columbia CNA Agar with 5% Sheep Blood (BD catalog #221353) and incubated at 42° C. Columbia CNA agar with 5% Sheep Blood was employed as it is a selective microbiological medium that is employed for the isolation of Gram-positive bacteria which would include those capable of sporulation. Following 24 hours incubation CNA plates were examined for colony morphology and selected colonies were re-struck onto fresh CNA plates and incubated as before. Following overnight incubation, colonies were selected from plates and inoculated into 4 ml of Histamine Minimal Medium supplemented with 10 mM histamine in individual wells of a 6-well tissue culture plate (Greiner Bio-One catalog #657 160) and placed into a 42° C. incubator.

Cultures were then sampled for histamine degradation and compared to a non-bacterial containing medium control well. Cultures showing greater than 50% degradation during an 8 day time course of culture were then plated onto fresh CNA plates, incubated at 42° C., and the following day individual colonies selected and subjected to MALDI-TOF for the identification of bacterial genus and species.

### 2. Use of UHPLC for Histamine Quantitation:

Nine hundred microliters of culture supernatants were collected at designated times from culture tubes and added to 100 μL of 2M perchloric acid in a 2 mL Eppendorf tube. The tube was then vortexed to mix the samples and then centrifuged at 15,000 rpm for 10 minutes at 4° C. to pellet the bacterial contents and provide a cell-free supernatant to be used for histamine quantitation. One-hundred microliters of cell-free supernatant was transferred into a 2 mL, 9 mm wide amber vial containing 900RL of MD-TM mobile phase.

To analyze on the UHPLC instrument, the quantitation was based on published methodology (Frattini V, Lionetti C. Histamine and histidine determination in tuna fish samples using high-performance liquid chromatography. Derivatization with omicron-phthalaldehyde and fluorescence detection or UV detection of “free” species. J Chromatogr A. 1998; 809(1-2):241-5; doi: 10.1016/s0021-9673(98)00157-5; Cicero A, Galluzzo F G, Cammilleri G, Pulvirenti A, Giangrosso G, Macaluso A, et al. Development of a Rapid and Eco-Friendly UHPLC Analytical Method for the Detection of Histamine in Fish Products. Int J Environ Res Public Health. 2020; 17(20); doi: 10.3390/ijerph17207453; and Vitali L, Valese A C, Azevedo M S, Gonzaga L V, Costa A C, Piovezan M, et al. Development of a fast and selective separation method to determine histamine in tuna fish samples using capillary zone electrophoresis. Talanta. 2013; 106:181-5; doi: 10.1016/j.talanta.2012.12.020, each herein incorporated by reference in their entirety). In brief, the amber vials were transferred to the carousel that was cooled to 4° C. in order of sequence determined by the sequence created in the Chromeleon 7 instrument program. The samples were analyzed using an Ultimate 3000 UHPLC system including electrochemical detector (ECDRS) and variable wavelength detector (VWD). ECDRS was set to 400 mV with a column temperature of 27° C. using a C18 Hypersil (150 mm length, 3 mm diameter, and 2.6 μm) column. VWD had two channels with wavelengths set to 210 and 280 nm. Flow rate was set to 0.6 mL/min using MD-TM from ThermoFisher (10% acetonitrile) and 10% IPA as a rear seal wash. All analyses were completed in the Chromeleon 7 instrument program by analyzing every peak of interest using analytical standards purchased from ThermoFisher Scientific. Results were transferred and calculated in Microsoft Excel and graphed using the Prism 8 statistical program (GraphPad, San Diego, CA).

3. Results from Histamine Feeding Trial

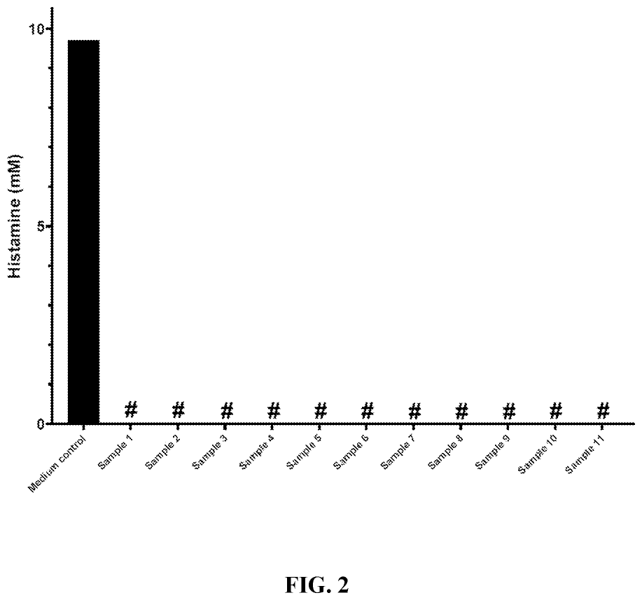

Samples were removed from the culture wells at the end of the culture period and were processed for histamine analysis using UHPLC technique. Additionally, samples were also plated on CNA agar with 5% Sheep Blood. The morphological appearance of bacterial growth after overnight incubation at 42° C. indicated the presence of only one bacterial species. This was confirmed and bacterial species identified by selection of a representative colony which was then subjected to MALDI-TOF analysis. All wells which evidenced 100% degradation of histamine as determined by UHPLC analysis were also shown by MALDI-TOF to contain Brevibacillus borstelensis. As shown in FIG. 2, 100% degradation of histamine was observed in wells which contained Gram-positive bacteria which belonged to the genus Brevibacillus.

FIG. 2 shows 5-day cultures of B. borstelensis isolated from chickens on a low and high histamine diet were capable of completely degrading the initially high level of histamine (10 mM) that was present at the initiation of culture. Each sample number in the Figure pertains to an isolate of B. borstelensis obtained from an individual chicken that belonged to Group 2 which received either the low or high histamine feeds of 5 or 15 mg of histamine per 100 grams of feed, respectively. In FIG. 2, “Medium Control” column indicates that Histamine Minimal medium supplemented with 10 mM histamine in the absence of added bacteria did not evidence any degradation of histamine over the time course. Cultures of B. borstelensis were able to achieve 100% degradation of histamine as shown in FIG. 2 by the symbol “#”.

These results suggest that to achieve this reduction of histamine, use of bacteria either as a probiotic or as a source of enzymes to use in, for example, animal feed in a sufficient amount of histamine degrading probiotics, such as 10⁴ to 10¹⁴ CFUs of a probiotic and/or from about 10¹ HDU to about 10¹⁰ HDU of an enzyme, may be administer to a subject. This would result in the decrease in histamine in the gut, thereby preventing the irregularities seen in inflammation and gut permeability by having a high level of histamine present. As such, the administration of a histamine degrading Brevibacillus probiotic would decrease or prevent the conditions associated with excess histamine.

### Example 3

To determine whether Bacillus spp. are capable of degrading histamine, additional in vitro trials were conducted.

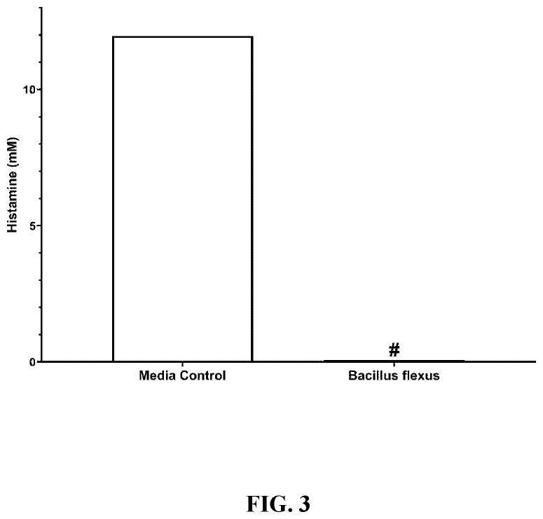

Bacillus flexus was cultured in ⅓rd concentration Luria-Bertani (LB) medium supplemented with 10 mM histamine. After 2 days, remaining histamine levels were measured using UHPLC methodology described in Example 2. As shown in FIG. 3, B. flexus degraded 100% of the histamine. The control (no bacteria added to medium), did not show any degradation of histamine.

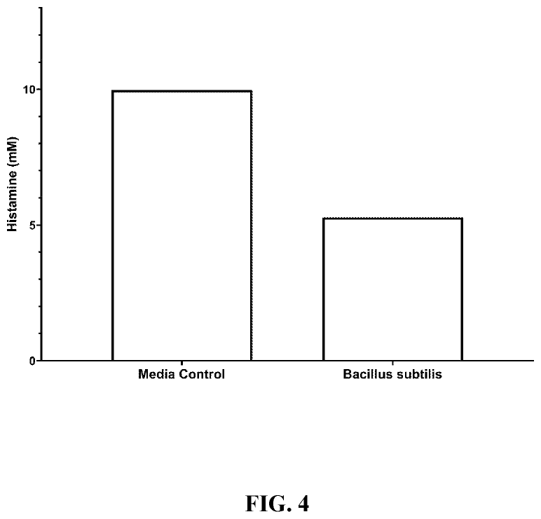

Bacillus subtilis was cultured in Histamine Minimal Medium (HMM) containing 10 mM histamine. After 2 days, remaining histamine levels were measured using UHPLC methodology described in Example 2. As shown in FIG. 4, B. subtilis degraded nearly 50% of the histamine. The control (no bacteria added to medium), did not show any degradation of histamine.

These results show that additional bacterial species, such as Bacillus spp., are capable of degrading histamine. As such, the administration of a histamine degrading Bacillus probiotic may decrease or prevent conditions associated with excess histamine.

### Example 4

To determine whether Enterococcus spp. are capable of degrading histamine, additional in vitro trials can be conducted.

Enterococcus spp. can be cultured in an appropriate medium, for example ⅓rd concentration Luria-Bertani (LB) medium supplemented with 10 mM histamine. After 2 days, remaining histamine levels can be measured using UHPLC methodology described in Example 2. It is expected that Enterococcus spp. will degrade histamine whereas the control (no bacteria added to medium) will not show any degradation of histamine.

These results can show that additional bacterial species, such as Enterococcus spp., are capable of degrading histamine. As such, the administration of a histamine degrading Enterococcus probiotic may decrease or prevent conditions associated with excess histamine.

What is claimed is:

1. A method of controlling histamine production in a subject comprising:
administering a probiotic composition comprising a Brevibacillus spp. and/or Bacillus spp. and optionally a histamine degrading enzyme to the subject; and
controlling histamine production within the subject.

2. The method of claim 1, wherein the Brevibacillus spp. is Brevibacillus borstelensis, Brevibacillus brevis, and/or Brevibacillus parabrevis.

3. The method of claim 1, wherein the Bacillus spp. is Bacillus flexus and/or Bacillus subtilis.

4. The method of claim 1, wherein the subject is a pig, chicken, duck, goose, turkey, guinea fowl, ostrich, pigeon, quail, and/or pheasant.

5. The method of claim 1, wherein the subject is a companion animal or aquaculture species.

6. The method of claim 1, wherein the subject is a human.

7. The method of claim 1, wherein the controlling of histamine is a reduction in the microbial production of histamine.

8. The method of claim 1, wherein the histamine-degrading enzyme is amine oxidase, and optionally wherein the enzyme is a purified enzyme.

9. The method of claim 1, wherein the Brevibacillus spp. and/or Bacillus spp. is a live microorganism, a dead and/or inactivated microorganism, a spore that is germinated into a histamine-degrader in the subject, or a mash incorporating a microorganism therein.

10. The method of claim 1, wherein the probiotic composition and optional histamine-degrading enzyme is neat, coated or encapsulated and administered in a feed or water source.

11. A method of preventing and/or treating an inflammatory disease and/or an immunological condition mediated by histamine in a subject, comprising:
administering a probiotic composition comprising a Brevibacillus spp. and/or Bacillus spp. and optionally a histamine degrading enzyme to the subject; and
controlling histamine production within the subject.

12. The method of claim 11, wherein the inflammatory disease and/or immunological condition is clostridial dermatitis, clostridial enteric disease, gut inflammation, necrotizing enterocolitis, neoplastic disease, and/or bacterial infection.

13. The method of claim 11, wherein the Brevibacillus spp. is Brevibacillus borstelensis, Brevibacillus brevis, and/or Brevibacillus parabrevis.

14. The method of claim 11, wherein the Bacillus spp. is Bacillus flexus and/or Bacillus subtilis.

15. The method of claim 11, wherein the probiotic composition comprises from about 10⁴ CFU to about 10¹⁴ CFU of the Brevibacillus spp. and/or Bacillus spp.

16. The method of claim 11, wherein the administration is oral or topical.

17. A probiotic composition, comprising:
a therapeutically effective amount of a Brevibacillus spp. and/or Bacillus spp.; and
a therapeutically acceptable excipient, additional functional ingredient, and/or a food/feed component.

18. The composition of claim 17, wherein the Brevibacillus spp. is Brevibacillus borstelensis, Brevibacillus brevis, and/or Brevibacillus parabrevis and the Bacillus spp. is Bacillus flexus and/or Bacillus subtilis.

19. The composition of claim 17, wherein therapeutically effective amount of the Brevibacillus spp. and/or Bacillus spp. is from about 10⁴ CFU to about 10¹⁴ CFU.

20. The composition of claim 17, wherein the additional functional ingredient is a histamine-degrading enzyme.
